(19) United States
(12) Patent Application Publication
(10) Pub. No.: US 2019/0199078 A1
(43) Pub. Date: Jun. 27, 2019

(54) Providing Strain Relief in Electrical Cable Assemblies

(71) Applicant: Biosense Webster (Israel) Ltd., Yokneam (IL)

(72) Inventor: Yinon Nisim Edrei, Zichron Yaakov (IL)

(21) Appl. No.: 15/855,244

(22) Filed: Dec. 27, 2017

(51) Int. Cl. H02G 15/007; H02G 15/18; H02G 1/00; A61B 18/14; A61B 5/042

(52) U.S. Cl. CPC H02G 15/007; H02G 15/18; A61B 2018/00178; A61B 18/1492; A61B 5/042; H02G 1/005

(57) ABSTRACT

A cable assembly includes a main section and at least first and second sub-sections. The main section includes multiple cables. The at least first and second sub-sections split from the main section at a junction point and include respective first and second subsets of the cables, the second sub-section is shorter than the first sub-section and has a larger tensile strength than the first sub-section.

### FIELD OF THE INVENTION

The present invention relates generally to electronic systems, and particularly to methods and systems for controlling strain relief in electrical cables.

### BACKGROUND OF THE INVENTION

Electrical cable assemblies are used, for example, for interconnecting among modules in electronic systems. Various mechanisms are known in the art for protecting cable assemblies from mechanical damage.

For example, U.S. Pat. No. 5,409,400 describes a shielding apparatus for an electrical connector. The apparatus comprises a front shell and back-shells that nest one with the other, short anchoring flanges and long anchoring flanges that interlock in recesses of a strain relief, and walls that interlock at a second location on the back-shells.

U.S. Pat. No. 5,763,832 describes a junction box connector and resulting assembly that facilitate the inclusion of a strain wire. The strain wire relieves the load placed upon the cable or flexible conduit and cable combination that extends between the junction box and another similar connection.

### SUMMARY OF THE INVENTION

An embodiment of the present invention that is described herein provides a cable assembly including a main section and at least first and second sub-sections. The main section includes multiple cables. The at least first and second sub-sections split from the main section at a junction point and include respective first and second subsets of the cables, the second sub-section is shorter than the first sub-section and has a larger tensile strength than the first sub-section.

In some embodiments, the second sub-section is configured to serve as a strain relief to at least the first sub-section. In other embodiments, the first sub-section is thinner than the second sub-section. In yet other embodiments, at least the first and second sub-sections are configured to electrically connect to a control console.

In an embodiment, the main section is configured to electrically connect to a medical probe. In another embodiment, the first subset of the cables is configured to conduct electro-potential (EP) signals between sensing electrodes of a medical probe and a processor. In yet another embodiment, the second subset of the cables is configured to conduct ablation signals between a signal generator and ablation electrodes of a medical probe.

In some embodiments, the cable assembly includes a strain relief element coupled to at least one of the first and second sub-sections. In other embodiments, the strain relief element has a tensile strength larger than of the first sub-section.

There is additionally provided, in accordance with an embodiment of the present invention, a method for producing a cable assembly, the method including producing a main section that includes multiple cables. At least first and second sub-sections that include respective first and second subsets of the cables are split up from the main section at a predefined junction point. The second sub-section is shorter than the first sub-section and has a larger tensile strength than the first sub-section.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a catheterization system in which the catheter cable is mechanically protected, in accordance with an embodiment of the present invention.

### DETAILED DESCRIPTION OF EMBODIMENTS

### Overview

Cable assemblies are used for connecting modules in various types of industrial electronic systems, such as in medical systems. A cable assembly typically comprises a cable terminated by connectors, which are configured to connect between the cable and a mating socket of a respective module. Some cable assemblies may comprise multiple sub-sections that split from a main section.

In principle, cable assemblies may be protected mechanically against excessive tensile strain, by incorporating a strain relief element in each sub-section of the cable assembly. A strain relief element protects the coupling between the sub-section and the respective connector from being ripped out when the cable is pulled, e.g., accidentally. Strain relief elements typically comprise a thick plastic or metal stripe, and thus may substantially increase the volume and cost of the cable assembly.

Embodiments of the present invention that are described hereinbelow provide improved techniques for reducing the number of strain relief elements in a cable assembly. In some embodiments, a cable assembly comprises a main section that comprises multiple cables, and multiple sub-sections that split from at least one junction point, each sub-section comprising a respective subset of the cables.

In some embodiments, the cable assembly is designed so that the shortest sub-section has the largest tensile strength (i.e., highest resilience to tearing) among all of the sub-sections, thereby serving as a strain relief element for protecting the other sub-sections of the respective junction point against tearing when the cable assembly is pulled. In some embodiments, the shortest sub-section eliminates the need for a strain relief element in a longer sub-section. In other embodiments, none of the sub-sections comprises a strain relief elements, and the shortest sub-section has a large tensile strength merely by virtue of the thickness or structure of its cables or insulation material.

In other embodiments, the cable assembly may incorporate a strain relief element, which is extended from the junction point and is configured to protect the shortest sub-section when a user pulls the cable assembly.

The disclosed techniques may assist in reducing the cost, complexity and bulk volume of cable assemblies in electronics systems, and are particularly important in protecting mechanically cable assemblies having a large number of sub-sections.

### System Description

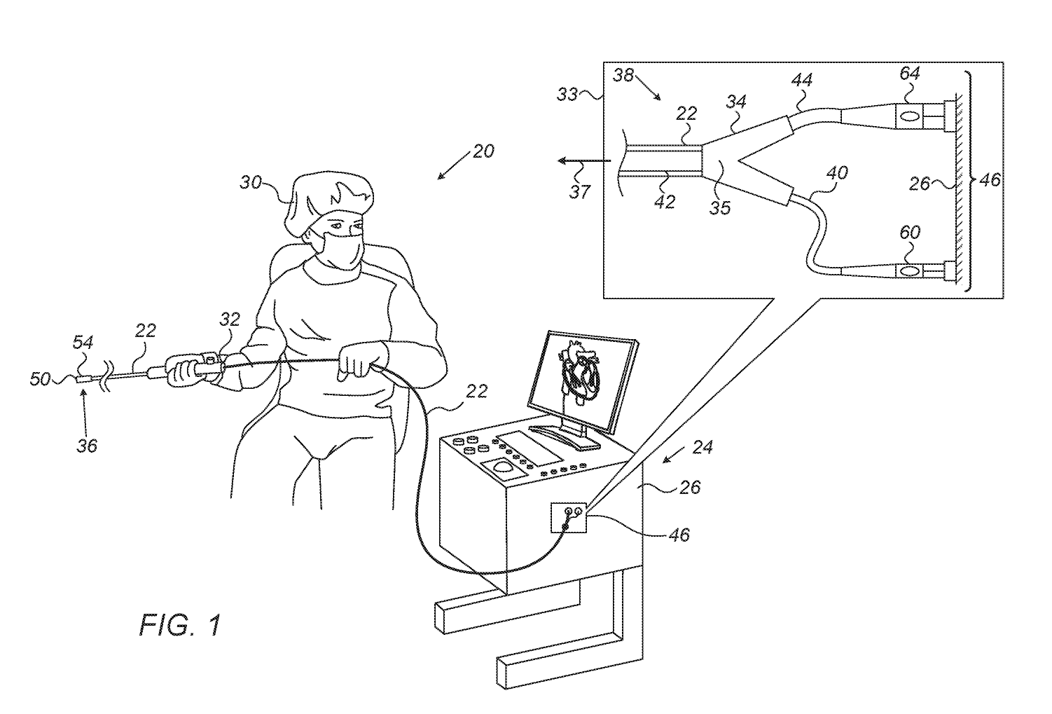

FIG. 1 is a schematic, pictorial illustration of a catheterization system 20, in accordance with an embodiment of the present invention. System 20 comprises a probe, in the present example a cardiac catheter 22.

In some embodiments, catheter 22 may be used for any suitable therapeutic and/or diagnostic purposes, such as for sensing electro-potential signals or for ablating tissue in a patient heart (not shown.)

In some embodiments, catheter 22 comprises a distal end assembly 36, which is coupled to the distal end of catheter 22. In some embodiments, assembly 36 comprise sensing electrodes 50 that may be used for electro-potential (EP) mapping of heart tissue, and ablation electrodes 54 for ablating tissue at a target location of the heart.

In some embodiments, system 20 comprises a control console 24 operated by a physician 30. Console 24 comprises a processor (not shown), typically a general-purpose computer, with suitable front end and interface circuits for receiving signals via catheter 22 and for controlling other components of system 20.

In some embodiments, console 24 comprises, instead of, or in addition to the processor, a general-purpose computer, which is programmed in software to carry out the functions described herein. The software may be downloaded to the computer in electronic form, over a network, for example, or it may, alternatively or additionally, be provided and/or stored on non-transitory tangible media, such as magnetic, optical, or electronic memory.

In an embodiment, physician 30 navigates distal end assembly 36 in the vicinity of the target location in the heart by manipulating catheter 22 using a manipulator 32 coupled to the proximal end of catheter 22.

### Strain Relief in Electrical Cable Assembly

Reference is now made to an inset 33. In some embodiments, catheter 22 comprises a cable assembly 38, which is configured to electrically connect, via a connection socket 46 in a panel 26, between components of catheter 22, such as distal end assembly 36, and the interface circuits of console 24.

Inset 33 is a side view of cable assembly 38 connected to panel 26 at connection socket 46. In some embodiments, cable assembly 38 comprises a main section 42 comprising multiple cables (not shown). In the example of FIG. 1, main section 42 extends between assembly 36 and a junction point, also referred to herein as a splitter 34. In an embodiment, splitter 34 splits main section 42 into sub-sections 40 and 44, which are connected to panel 26 via connectors 60 and 64 of connection socket 46, respectively.

In some embodiments, some of the cables of assembly 38 are extended between electrodes 50 and console 24, via sub-section 40. The other cables of catheter 22, such as the cables coupled to electrodes 54, and typically additional cables of catheter 22, are connected to console 24 via sub-section 44.

In an embodiment, sub-section 40 is typically thinner than sub-section 44 as it comprises a smaller number of cables and/or thinner cables compared to the cables of sub-section 44. For example, sub-section 40 may have three cables connected to three respective electrodes 50, each cable is configured to conduct low currents, such as less than 10 μAmperes, of EP signals, and therefore has an exemplary diameter of 0.2 mm. Sub-section 44 may comprise, for example, 10 cables, some of which are configured to conduct ablation signals of 1 Ampere and therefore have higher thickness, e.g. 0.5 mm. Furthermore, sub-section 44 may comprise additional cables, such as cables configured to conduct signals produced by a thermocouple, having exemplary diameter of 0.5 mm.

In some embodiments, sub-sections 40 and 44 may have typical diameters of 4 mm and 8 mm, respectively. In some embodiments, sub-section 44 is more resilient to tearing than sub-section 40, because sub-section 44 is thicker than sub-section 40 and therefore has larger tensile strength.

In some embodiments, sub-section 44 is shorter than sub-section 40, for example, the length of sub-section 40, measured between a splitting point 35 and connector 60, is about 250 mm, whereas the length of sub-section 44, measured between splitting point 35 and connector 64, is about 150 mm. In an operative mode, when an operator (e.g., physician 30) pulls catheter 22 in a direction marked by an arrow 37, he/she applies a tensile stress to cable assembly 38. In response to the applied tensile stress, sub-section 44 is stretched, whereas sub-section 40 remains loose as shown in inset 33. In this embodiment, sub-section serves as a strain relief by absorbing the applied tensile stress, thereby protecting sub-section 40 from tearing. Furthermore, in this embodiment, sub-section 44 may be used instead of a strain relief element to protect sub-section 40 from tearing.

In some embodiments, cable assembly 38 may comprise a stain relief element (not shown) typically coupled to sub-section 44, for example, between splitter 34 and connector 64, and having tensile strength (i.e., resilience to tearing) larger than of sub-section 44. In some embodiments, the strain relief element may comprise a plastic sleeve, or a metal wire, or any other suitable configuration, and typically improves the resilience to tearing larger than of sub-section 44.

In other embodiments, the strain relief element may be coupled to sub-section 40. In these embodiments, the strain relief element may be shorter than sub-section 40, so as to protect only sub-section 40 from tearing. Alternatively or additionally, the strain relief element may be shorter than sub-section 44, so as to protect both sub-sections 40 and 44 from tearing.

The example of FIG. 1 refers to a specific configuration of a cable assembly. This configuration, however, is chosen purely for the sake of conceptual clarity. In alternative embodiments, the disclosed techniques can be used, mutatis mutandis, in various other configurations. For example, a cable assembly may comprise more than two sub-sections, such that the shortest sub-section may have the largest tensile strength among all the sub-sections of the cable assembly. In other words, in a cable assembly having multiple sub-sections extending from a predefined junction point, for each sub-section, the smaller the tensile strength, the longer the length of the sub-section. As described above, at least one of the sub-sections, typically, but not necessarily, the shortest sub-section, may have a strain relief element coupled thereto.

Although the embodiments described herein mainly address cable assemblies of medical systems, the methods and systems described herein can also be used in other applications, such as in any electronic system having multiple modules, as well as in cable assemblies interconnecting between electronic systems.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A cable assembly, comprising:
a main section comprising multiple cables; and
at least first and second sub-sections, which split from the main section at a junction point and comprise respective first and second subsets of the cables, wherein the second sub-section is shorter than the first sub-section and has a larger tensile strength than the first sub-section.

2. The cable assembly according to claim 1, wherein the second sub-section is configured to serve as a strain relief to at least the first sub-section.

3. The cable assembly according to claim 1, wherein the first sub-section is thinner than the second sub-section.

4. The cable assembly according to claim 1, wherein at least the first and second sub-sections are configured to electrically connect to a control console.

5. The cable assembly according to claim 1, wherein the main section is configured to electrically connect to a medical probe.

6. The cable assembly according to claim 1, wherein the first subset of the cables is configured to conduct electro-potential (EP) signals between sensing electrodes of a medical probe and a processor.

7. The cable assembly according to claim 1, wherein the second subset of the cables is configured to conduct ablation signals between a signal generator and ablation electrodes of a medical probe.

8. The cable assembly according to claim 1, and comprising a strain relief element coupled to at least one of the first and second sub-sections.

9. The cable assembly according to claim 8, wherein the strain relief element has a tensile strength larger than of the first sub-section.

10. A method for producing a cable assembly, the method comprising:
producing a main section comprising multiple cables; and
splitting from the main section, at a predefined junction point, at least first and second sub-sections comprising respective first and second subsets of the cables, wherein the second sub-section is shorter than the first sub-section and has a larger tensile strength than the first sub-section.

11. The method according to claim 10, wherein splitting the at least first and second sub-sections comprises producing the second sub-section as a strain relief to at least the first sub-section.

12. The method according to claim 10, wherein splitting the at least first and second sub-sections comprises producing the first sub-section thinner than the second sub-section.

13. The method according to claim 10, wherein splitting the at least first and second sub-sections comprises electrically connecting at least the first and second sub-sections to a control console.

14. The method according to claim 10, wherein producing the main section comprises electrically connecting the main section to a medical probe.

15. The method according to claim 10, wherein the first subset of the cables is for conducting electro-potential (EP) signals between sensing electrodes of a medical probe and a processor.

16. The method according to claim 10, wherein the second subset of the cables is for conducting ablation signals between a signal generator and ablation electrodes of a medical probe.

17. The method according to claim 10, and comprising coupling a strain relief element to at least one of the first and second sub-sections.

18. The method according to claim 17, wherein the strain relief element has a tensile strength larger than of the first sub-section.
